(19) United States
(12) Patent Application Publication
(10) Pub. No.: US 2014/0088585 A1
(43) Pub. Date: Mar. 27, 2014

(54) CATHETER HAVING RIB AND SPINE STRUCTURE SUPPORTING MULTIPLE ELECTRODES FOR RENAL NERVE ABLATION

(71) Applicant: BOSTON SCIENTIFIC SCIMED, INC., Maple Grove, MN (US)

(72) Inventors: DAVID M. HILL, LONG LAKE, MN (US); JASON P. HILL, BROOKLYN PARK, MN (US)

(73) Assignee: BOSTON SCIENTIFIC SCIMED, INC., MAPLE GROVE, MN (US)

(21) Appl. No.: 14/037,125

(22) Filed: Sep. 25, 2013

Related U.S. Application Data
(60) Provisional application No. 61/705,925, filed on Sep. 26, 2012.

(51) Int. Cl. A61B 18/18

(52) U.S. Cl. CPC A61B 18/18
USPC 606/33

(57) ABSTRACT

A catheter for ablating target tissue from a location within a body vessel includes an ablation region that is configured to transition from a first substantially straight configuration to a second configuration having a two-dimensional or three-dimensional shape. The ablation region may include a plurality of ablation elements that may be distributed along a length of the ablation region such that when the ablation region is in the second configuration, the ablation elements may be placed in closer proximity to the target tissue. Additionally, when the ablation region is in the second configuration, the ablation elements may achieve circumferential coverage of the body lumen or blood vessel, and as such, may be capable of ablating the target tissue at multiple locations along the length and around a circumference of the body lumen or vessel in a single step.

### CROSS-REFERENCE TO RELATED APPLICATIONS

This Application claims the benefit under 35 USC §119 of U.S. Provisional Application No. 61/705,925, filed on Sep. 26, 2012, the entirety of which is incorporated herein by reference.

### TECHNICAL FIELD

This disclosure generally relates to percutaneous and intravascular devices for nerve modulation and/or ablation.

### BACKGROUND

Certain treatments may require the temporary or permanent interruption or modification of select nerve function. One example treatment is renal nerve ablation which is sometimes used to treat conditions related to hypertension and/or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many bodily tissues such as nerves, including renal nerves, brain tissue, cardiac tissue and the tissue of other body organs are in close proximity to blood vessels or other body cavities and, thus, can be accessed percutaneously or intravascularly through adjacent blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

Because the nerves are hard to visualize, treatment methods employing such energy sources have tended to apply the energy as a generally circumferential ring to ensure that the nerves are modulated. However, such a treatment may result in thermal injury to the vessel wall near the electrode and other undesirable side effects such as, but not limited to, blood damage, clotting, weakened vessel wall, and/or protein fouling of the electrode.

### BRIEF SUMMARY

Some illustrative embodiments pertain to an intravascular catheter for modulating and/or ablating renal nerves which includes an elongated catheter body having an ablation region. The ablation region can include a flexible portion having a plurality of slots formed therein defining at least one spine extending along a length of the flexible portion and a plurality of ribs extending away from the spine such that the flexible portion is configured to transition from a first configuration suitable for delivery of the catheter to a second configuration having at least one bend, curve or turn suitable for ablating renal nerves. Additionally, the catheter can include at least one conductor extending within the elongated catheter body; two or more ablation elements coupled to the conductor extending within the elongated catheter body and located along the ablation region; and an actuation member coupled to the ablation region for transitioning the ablation region from the first configuration to the second configuration. In some embodiments, the two or more ablation elements are electrodes, wherein each electrode is configured to deliver sufficient RF energy so as to ablate renal nerves.

Some illustrative embodiments pertain to a method of ablating target nerve tissue from a location within a body vessel which includes delivering an intravascular catheter to a location within the body vessel adjacent the target nerve tissue. The catheter can include: an elongated catheter body having an ablation region configured to transition from a first configuration suitable for delivery of the catheter to a second configuration for ablating target tissue in a circumferential pattern along a length of the body vessel; at least one electrical conductor extending within the elongated catheter body; and a plurality of ablation elements located along the ablation region and coupled to the conductor extending within the elongated catheter body. Additionally, the methods can include transitioning the ablation region from the first configuration to the second configuration and delivering sufficient energy via the ablation elements positioned along the ablation region, wherein the target renal nerve tissue is ablated in a substantially circumferential pattern along the length of the body vessel.

The preceding summary is provided to facilitate an understanding of some of the innovative features unique to the present disclosure and is not intended to be a full description. A full appreciation of the disclosure can be gained by taking the entire specification, claims, drawings, and abstract as a whole.

### BRIEF DESCRIPTION OF THE DRAWINGS

The disclosure may be more completely understood in consideration of the following detailed description of various illustrative embodiments of the disclosure in connection with the accompanying drawings, in which:

FIG. 1 is a schematic view of an illustrative catheter deployed in a patient's renal artery at a location adjacent to a renal nerve;

FIG. 2 is a schematic view illustrating the location of the renal nerves relative to the renal artery;

FIGS. 3A and 3B are schematic, partially cut away side views of an illustrative catheter;

FIGS. 4A-4F are close-up, schematic views of several illustrative ablation regions of an exemplary catheter;

FIGS. 5A-5D are schematic views of several illustrative ablation regions of an exemplary catheter in a second configuration; and

FIGS. 6A-6B are partial, cross-sectional side views of an ablation region of an exemplary catheter disposed within a body lumen.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the disclosure.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used in connection with other embodiments whether or not explicitly described unless clearly stated to the contrary.

While the devices and methods described herein are discussed relative to renal nerve modulation through a blood vessel wall, it is contemplated that the devices and methods may be used in other applications where nerve modulation and/or ablation are desired. The term modulation refers to ablation and other techniques that may alter the function of affected nerves and other tissue such as brain tissue or cardiac tissue.

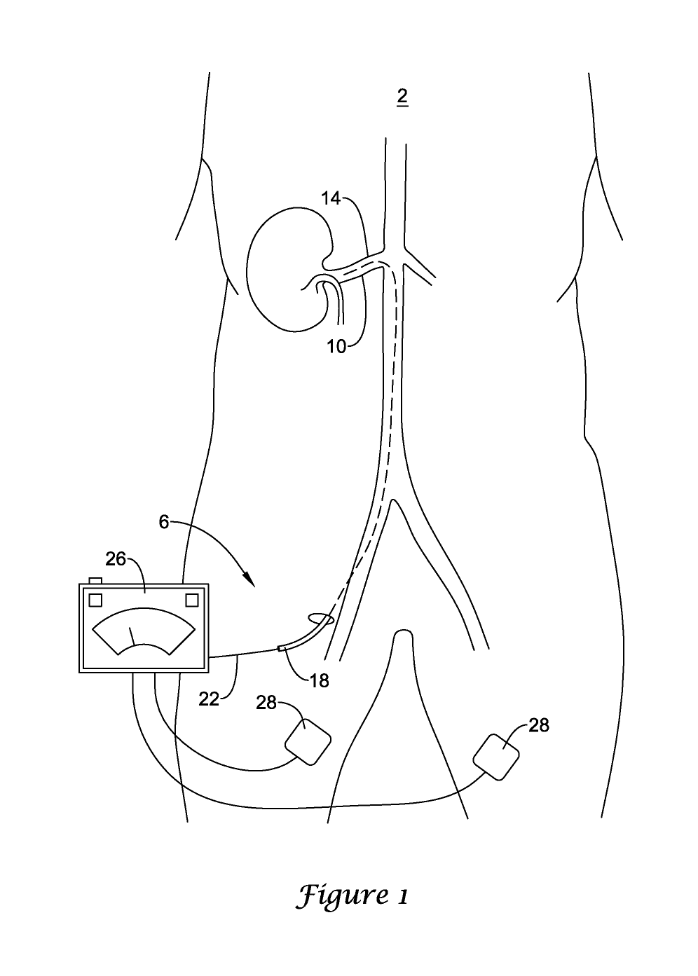

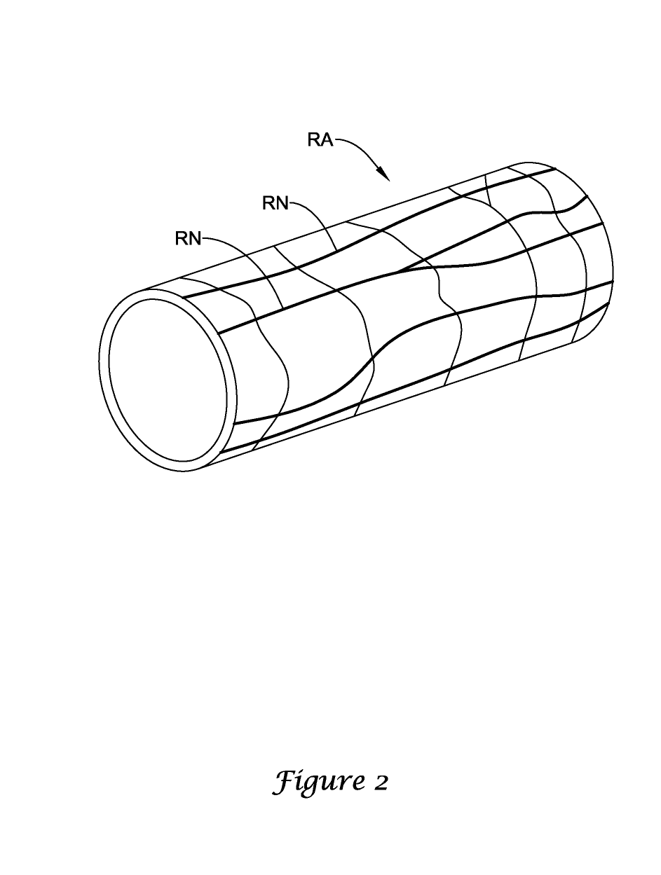

FIG. 1 is a schematic view of an exemplary renal nerve modulation system 6 disposed within a portion of a patient's renal system 2. FIG. 2 illustrates a portion of the renal anatomy in greater detail. The renal anatomy includes renal nerves RN extending longitudinally along the lengthwise dimension of renal artery RA and generally within or near the adventitia of the artery. The human renal artery wall is typically about 1 mm thick of which about 0.5 mm is the adventitial layer. As will be seen in the figure, the circumferential location of the nerves at any particular axial location may not be readily predicted. Renal nerves are difficult to visualize in situ. As such, treatment methods may desirably rely upon ablating multiple sites to ensure nerve modulation.

According to various illustrative embodiments, system 6 includes an intravascular, renal ablation catheter 18 and one or more conductor(s) 22 for providing power to catheter 18. A proximal end of conductor(s) 22 is connected to a control and power element 26, which supplies necessary electrical energy to activate one or more electrodes disposed along an ablation region at or near a distal end of catheter 18. When suitably activated, the electrodes are capable of ablating adjacent tissue. In some cases, a temperature sensing wire such as, for example, a thermocouple may also be used at each electrode. The terms electrode and electrodes may be considered to be equivalent to elements capable of ablating adjacent tissue in the disclosure which follows. In some instances, system 6 can include return electrode patches 28 that may be applied to the patient's legs or at another conventional location on the patient's body to complete the circuit.

In some embodiments, control and power element 26 includes monitoring elements to monitor parameters such as power, temperature, voltage, amperage, impedance, pulse size and/or shape and other suitable parameters. The monitoring elements may include sensors mounted along catheter 18, as well as suitable controls for performing a desired procedure. In some embodiments, control and power element 26 control the one or more electrodes located in an ablation region of the catheter 18, as will be described in more detail below. In some embodiments, the one or more electrodes include one or more radio frequency (RF) electrodes. The electrode(s) may be configured to operate at a frequency of approximately 460 kHz. It is contemplated that any desired frequency in the RF range may be used, for example, from 450-500 kHz. It is further contemplated that additional and/or other ablation devices may be used as desired, for example, but not limited to resistance heating, ultrasound, microwave, and laser devices, and these devices may require that power be supplied by the power element 26 in a different form.

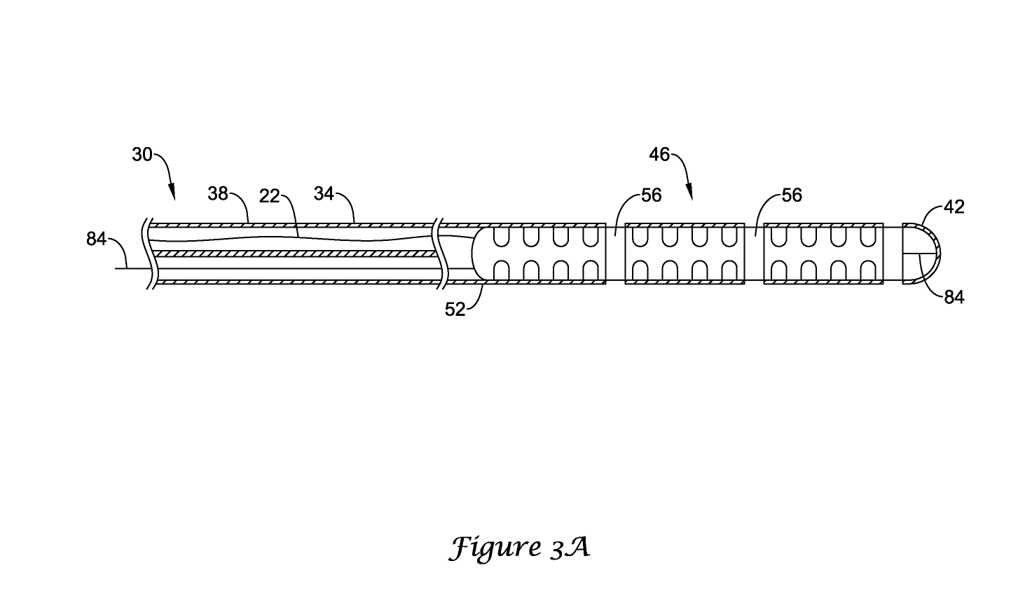

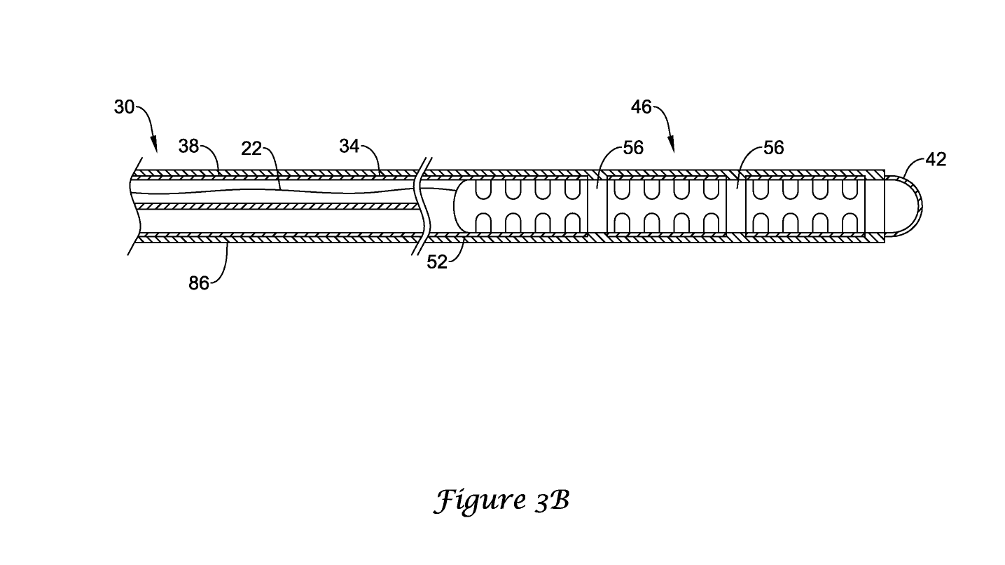

FIGS. 3A and 3B are partial, cross-sectional side views of an intravascular nerve ablation catheter 30 according to various embodiments as described herein. In some embodiments, intravascular nerve ablation catheter 30 is a renal nerve ablation catheter for ablating the renal nerves at one or more locations along a length of the renal nerves from a location within the renal artery. More particularly, intravascular nerve ablation catheter 30 can be a renal nerve ablation catheter for ablating the renal nerves at one or more locations around a circumference and along a length of the renal artery. As shown in FIGS. 3A and 3B, catheter 30 includes an elongated catheter body 34 that extends from a proximal end 38 to a distal end 42 of the catheter 30. In some cases, catheter body 34 may take the form of a metallic and/or polymer tubular body and may include visualization (e.g., marker bands) and/or reinforcing structures (e.g., braids, coils, etc.) commonly used for catheter bodies. Catheter body 34 may also include an additional lumen for delivery of a contrast dye to facilitate visualization of catheter 30 and/or artery when in use.

Catheter 30 also includes an ablation region 46 located at or near a distal region 52 of the catheter body 34. In some cases, the ablation region 46 may include the distal end 42 of the catheter body 34, but this is not required. As shown in FIGS. 3A and 3B, the ablation region 46 includes one or more ablation elements 56 that are configured to ablate target tissue at or near a target site within the patient's body. The one or more ablation elements 56 can be electrodes. For example, in one embodiment, the ablation elements 56 are RF electrodes that are configured to deliver sufficient RF energy so as to ablate nerve tissue from a location within an adjacent body lumen such as an artery or other blood vessel. The ablation elements 56 may be configured to ablate the renal nerve from a location within the renal artery. The ablation elements 56 are coupled to one or more electrical conductors 22 extending with the catheter body 34 from the proximal end 38 of the catheter 30 where they may be electrically coupled to the control and power element 26 (see, for example, FIG. 1). In certain cases where multiple ablation elements may be employed, each individual ablation element may be individually coupled to an electrical conductor extending within the catheter body 34 in a one to one manner such that individual ablation elements may be selectively activated and/or controlled by the control and power element 26.

According to various embodiments, the ablation region 46 of catheter 30 is flexible such that the ablation region 46 including the one or more ablation elements 56 may be more easily positioned near the target tissue such that catheter 30 may be capable of ablating the target tissue while minimizing damage to non-target tissue. For example, the ablation region 46 may be sufficiently flexible such that it is configured to transition from a first configuration suitable for delivery of catheter 30 to a position near the target tissue to a second configuration suitable for ablating the target tissue such as, for example, renal nerve tissue. In the first configuration, the ablation region 46 is substantially straight such that catheter 30 including the ablation region 46 may be delivered to a location in a body lumen or vessel adjacent the target tissue. In the second configuration, the ablation region 46 has a two-dimensional or three-dimensional shape including at least one bend, turn, or curve such that at least a portion of the ablation region 46 may be positioned in closer proximity to the target tissue for ablation.

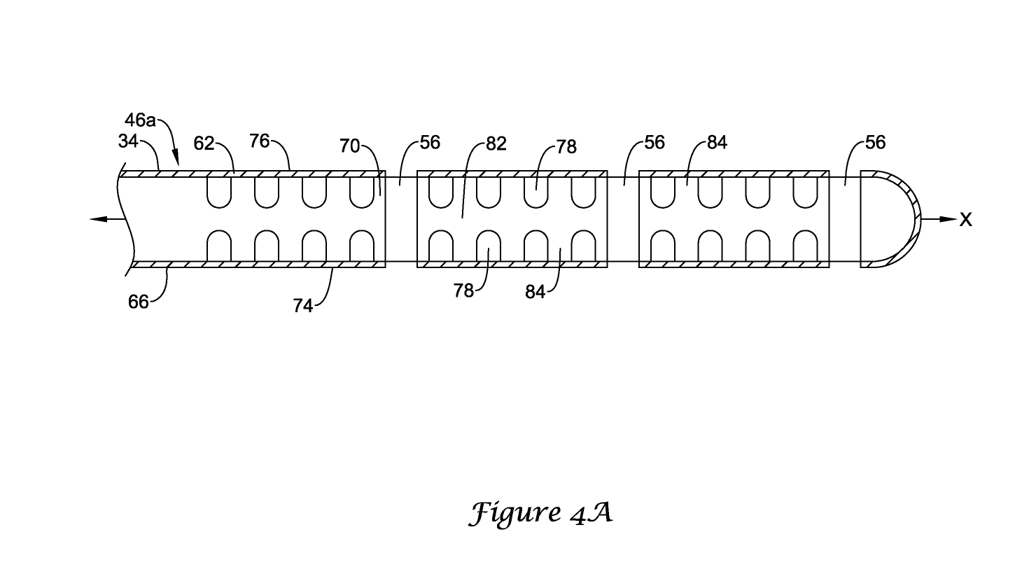

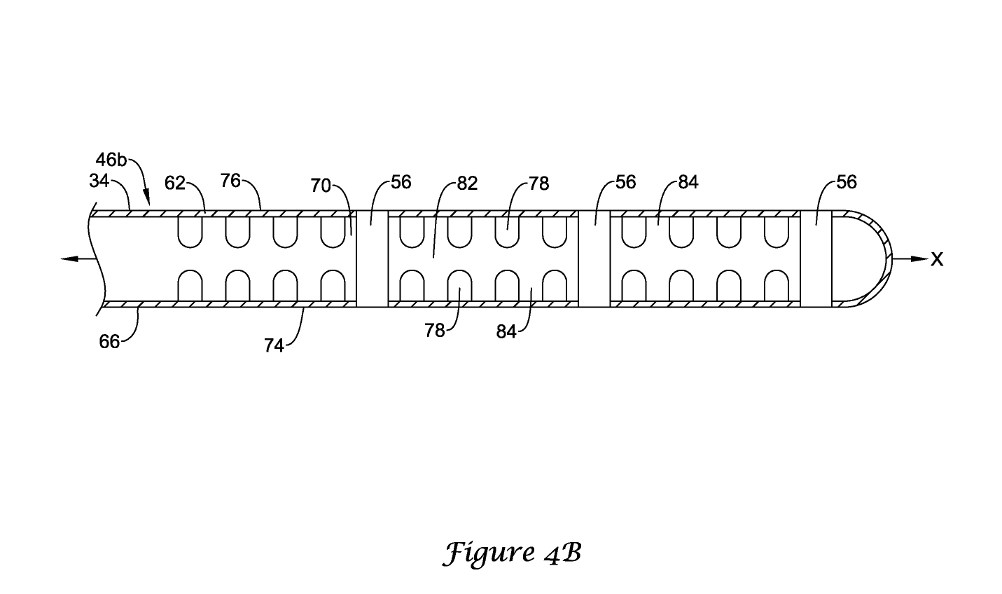

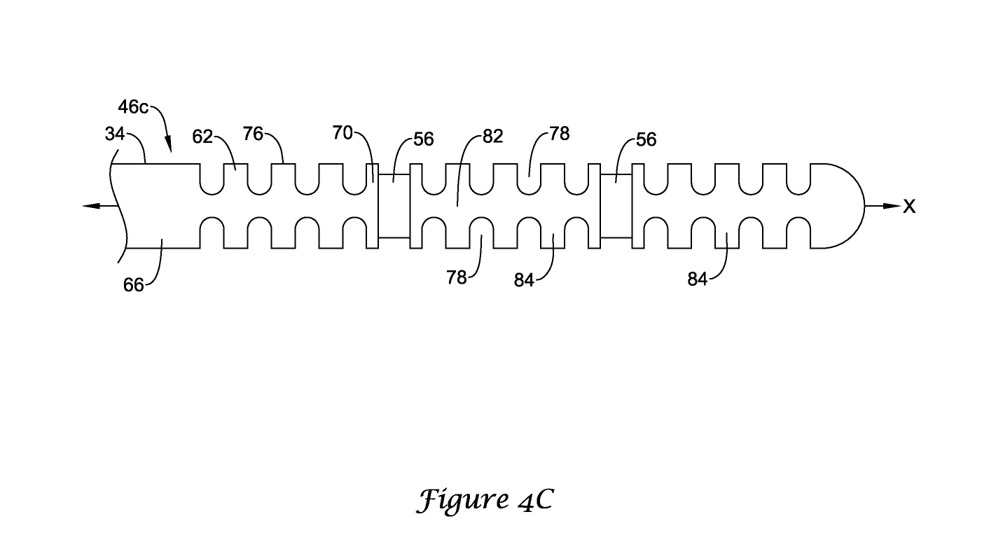

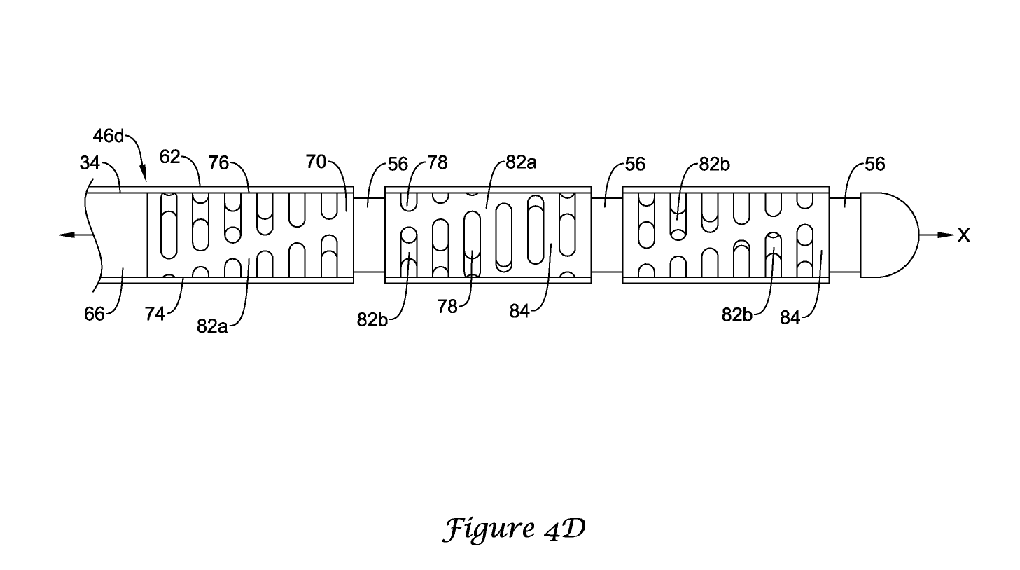

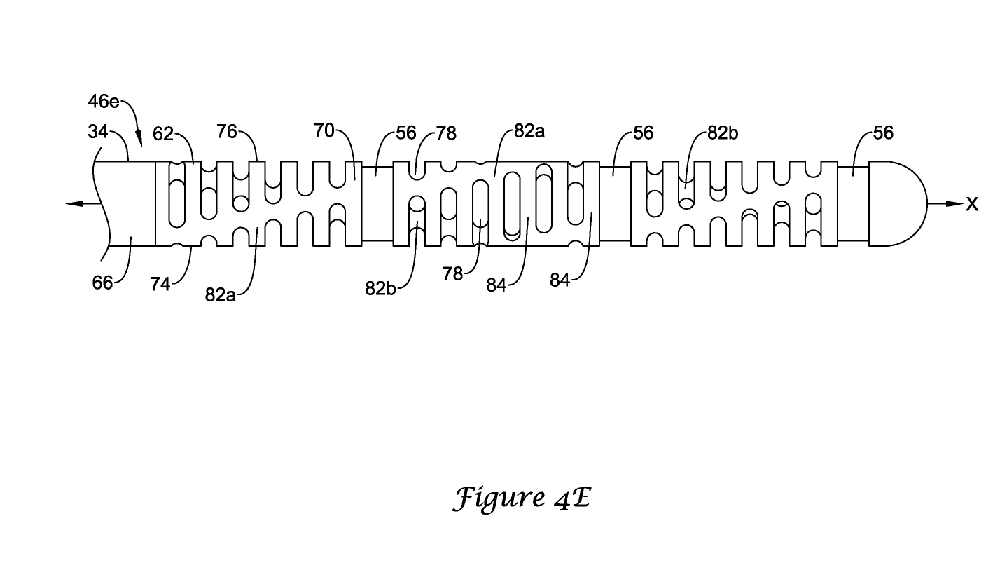

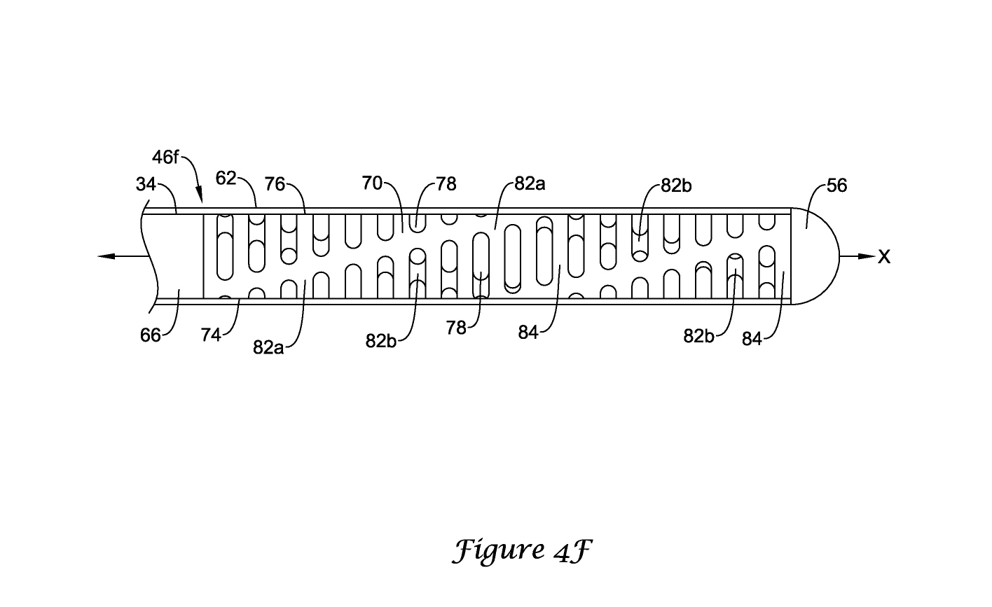

FIGS. 4A-4F are close-up, schematic views of several illustrative ablation regions 46a-46f that may be incorporated into a catheter body 34 of an exemplary catheter such as, for example, catheter 30 as described herein. As shown in the figures, each of the illustrative ablation regions 46a-46f includes a flexible portion 66 including a tubular member 70. The flexible portion 66, including tubular member 70 forms at least part of each of the ablation regions 46a-46f, as shown. In some cases, as shown in the illustrative examples of FIGS. 4A, 4B, 4D, and 4F, the tubular member 70 is a separate member from the catheter body 34, and may be disposed under at least one outer layer of insulative material 74 forming an outer surface 76 of the catheter body 34. In other cases, the tubular member 70 forms a part of the catheter body 34 including the outer surface 76 of the catheter body 34, as shown in the illustrative examples of FIGS. 4C and 4E.

In some cases, as shown, the tubular member 70 includes a plurality of cuts, slits, and/or slots 78 formed therein (collectively referred to herein as “slots”), thereby increasing the overall flexibility of the flexible portion 66 of each of the ablation regions 46a-46f. Slots 78 may be formed by methods such as micro-machining, saw-cutting (e.g., using a diamond grit embedded semiconductor dicing blade), electrical discharge machining, grinding, milling, casting, molding, chemically etching or treating, or other known methods, and the like. In such embodiments, the structure of the tubular member 70 is formed by cutting and/or removing portions of the tube to form slots 78. Some examples of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference for all purposes. Some examples of etching processes are described in U.S. Pat. No. 5,106,455, the entire disclosure of which is herein incorporated by reference for all purposes. In still some embodiments, slots 78 are formed in tubular member 70 using a laser cutting process.

Various arrangements and configurations are contemplated for slots 78 formed in the tubular member 70. For example, in some embodiments, at least some, if not all of slots 78 may be disposed at the same or a similar angle with respect to the longitudinal axis x of the tubular member 70. As shown in the illustrative embodiments of FIGS. 4A-4F, slots 78 are disposed at an angle that is perpendicular, or substantially perpendicular, and/or can be characterized as being disposed in a plane that is normal to the longitudinal axis x of tubular member 70. However, in other embodiments, slots 78 may be be disposed at an angle that is not perpendicular, and/or can be characterized as being disposed in a plane that is not normal to the longitudinal axis x of the tubular member 70. Additionally, a group of one or more slots 78 may be disposed at different angles relative to another group of one or more slots 78. The distribution and/or configuration of slots 78 may also include any of those disclosed in U.S. Pat. Publication No. U.S. 2004/0181174, which is incorporated by reference herein in its entirety for all purposes. These are just some examples.

Slots 78 are provided to enhance the flexibility of the tubular member 70 while still allowing for suitable torque transmission characteristics. Slots 78 can be formed such that one or more rings and/or tube segments interconnected by one or more segments and/or beams that are formed in the tubular member 70. Such tube segments and/or beams may include portions of the tubular member 70 that remain after slots 78 are formed in the tubular member 70. Such an interconnected structure may act to maintain a relatively high degree of torsional stiffness, while maintaining a desired level of lateral flexibility. In some embodiments, some adjacent slots 78 can be formed such that they include portions that overlap with each other about the circumference of the tubular member 70. In other embodiments, some adjacent slots 78 can be disposed such that they do not necessarily overlap with each other, but are disposed in a pattern that provides the desired degree of lateral flexibility.

Additionally, slots 78 may be arranged along the length of, or about the circumference of, the tubular member 70 to achieve desired properties. For example, adjacent slots 78 or groups of slots 78 can be arranged in a symmetrical pattern, such as being disposed essentially equally on opposite sides about the circumference of the tubular member 70, or can be rotated by an angle relative to each other about the axis of the tubular member 70. Additionally, adjacent slots 78, or groups of slots 78, may be equally spaced along the length of the tubular member 70, or can be arranged in an increasing or decreasing density pattern, or can be arranged in a non-symmetric or irregular pattern. Other characteristics, such as slot size, slot shape, and/or slot angle with respect to the longitudinal axis of tubular member 70, can also be varied along the length of the tubular member 70 in order to vary the flexibility or other properties.

In some embodiments, slots 78 may be formed in groups of two, three, four, five, or more slots 78, which may be located at substantially the same location along the axis of the tubular member 70. Within the groups of slots 78, there may be included slots 78 that are equal in size such that they may span the same circumferential distance around the tubular member 70. Additionally, in some embodiments, at least some slots 78 in a group may be unequal in size such that they span a different circumferential distance around tubular member 70. Longitudinally adjacent groups of slots 78 may have the same or different configurations. For example, some embodiments of the tubular member 70 include slots 78 that are equal in size in a first group and then unequally sized in an adjacent group.

In some cases, as shown in the illustrative embodiments of FIGS. 4A-4C, a plurality of slots 78 defines at least one spine 82 extending along a length of the ablation region 46 and a plurality of ribs 84 extending away from the spine 82. The spine 82 can be the portion of the tubular member 70 that remains after the slots 78 are formed and may, in some cases, extend parallel to the longitudinal axis x of the tubular member 70. In other embodiments, as shown in FIGS. 4D-4F, a plurality of slots 78 defines at least two spines 82a, 82b extending along a length of the ablation region 46 and a plurality of ribs extending between the two spines 82a, 82b from a first spine 82a to a second spine 82b. In some cases, the first spine 82a and the second spine 82b are disposed on opposite sides of the tubular member 70. More particularly, in some cases, the first spine 82a and 82b are located approximately 180 degrees opposite to one another on opposite sides of the tubular member 70. In still other embodiments, as shown in FIGS. 4D-4F, the spines 82a and 82b define an elongated spiral or helix along the length of the tubular member 70.

According to some embodiments, as shown in FIGS. 4A-4F, one or more ablation elements or electrodes 56 can be distributed along a length of each of the ablation regions 46a-46f including a distal end of the catheter body 34. The electrodes 56 may extend at least partially around an outer circumference of the catheter body 34. For example, in some embodiments, the electrodes 56 extend from about 45 degrees to about 225 degrees and more particularly, from about 90 degrees to about 180 degrees about the outer circumference of the catheter body 34. In other embodiments, the electrodes 56 extend from about 180 degrees to about 360 degrees about the outer circumference of the catheter body 34. In some embodiments, the electrodes 56 are recessed from an outer surface 62 of the catheter body 34 as shown in FIGS. 4A and 4C-4E. In another embodiment, as shown in FIG. 4B, the electrodes 56 have an electrode surface that is substantially planar with the outer surface 62 of the catheter body 34. Additionally, the electrodes 56 may have a thin layer of insulative material covering at least a portion of the outer surface of each of the electrodes 56, and may be sometimes referred to as “insulated wall-contact” electrodes.

As discussed herein, each of the ablation regions 46a-46f are sufficiently flexible such that they are capable of transitioning from a first configuration suitable for delivery of a catheter (e.g. catheter 30) to a location within a body lumen or vessel adjacent to the target nerve tissue to a second configuration suitable for ablating target tissue from the location within the adjacent body lumen or vessel using the multiple electrodes 56. The electrodes 56 are distributed along a length of each of the ablation regions 46a-46e such that when each of the ablation regions 46a-46e are in a second configuration, the electrodes 56 may achieve complete circumferential coverage of the body lumen or blood vessel while spaced apart longitudinally along its length. As such, when the ablation regions 46a-46e are in the second configuration, the electrodes 56 may be capable of ablating the nerves at multiple locations along the length and around a circumference of the body lumen without the need for repositioning the catheter (e.g. catheter 30) in the body lumen or vessel adjacent the target tissue.

In other embodiments, a single electrode 56 may be located along the ablation region 46f of the catheter body 34. In one embodiment, as shown in FIG. 4F, a single electrode 56 is located at a distal end of the catheter body 34 including ablation region 46f. The electrode 56 may be cylindrical and, in some cases, may include a hemispherical electrode tip. Additionally, an outer diameter of the electrode 56 may be substantially equal to the outer diameter of the catheter body 34 such that the outer surface of the electrode 56 does not protrude beyond the outer surface of the catheter body. Like ablation regions 46a-46e, ablation region 46f is sufficiently flexible such that it is capable of transitioning from a first configuration suitable for delivery of the catheter to a location within a body lumen or vessel adjacent the target nerve tissue to a second configuration suitable for facilitating ablation of the target tissue from the location within the adjacent body lumen or vessel using the single electrode 56. In some cases, the ablation region 46f may be configured to transition from a substantially straight configuration suitable for delivery of the catheter into the body lumen to a substantially sinusoidal second configuration suitable to position the electrode 56 located at the distal end of the catheter body 34 adjacent the target tissue for ablation.

Referring now back to FIGS. 3A and 3B, catheter 30 can include one or more actuation members 84, 86 that may be used to transition the ablation region 46 from the first configuration to a second configuration. In one embodiment, as shown in FIG. 3A, the actuation member 84 is a pull wire 84 that is coupled to a distal end of the ablation region 46 and, in some cases, that is coupled to a distal end 42 of the catheter body 34, as shown in FIG. 3A. The pull wire 84 extends in a proximal direction from a distal end 42 of the catheter body 34 to a location outside of the catheter body and that is accessible to a user. In use, a user can transition the ablation region 46 from the first configuration to the second configuration by pulling the pull wire 84 in a proximal direction (e.g.

toward the user). In one embodiment, the ablation region 46 can be transitioned back from the second configuration to the first configuration by pushing or releasing the pull wire 84 in a distal direction.

In another embodiment, as shown in FIG. 3B, the actuation member 86 is a sheath 86 that is disposed over at least the ablation region 46 of catheter 30. The sheath 86 extends in a proximal direction from a location near a distal end 42 of the catheter body 34 to a location outside of the catheter body 34 such that it may be accessible to the user. The sheath 86 retains the ablation region 46 in the first configuration during delivery of the catheter to a region in a body lumen or vessel adjacent the target tissue. In use, a user can transition the ablation region 46 from the first configuration to the second configuration by retracting the sheath 86 in a proximal direction to expose the ablation region 46. In this embodiment, the ablation region 46 is configured to automatically transition or expand from the first configuration to the second configuration upon retraction of the sheath 86.

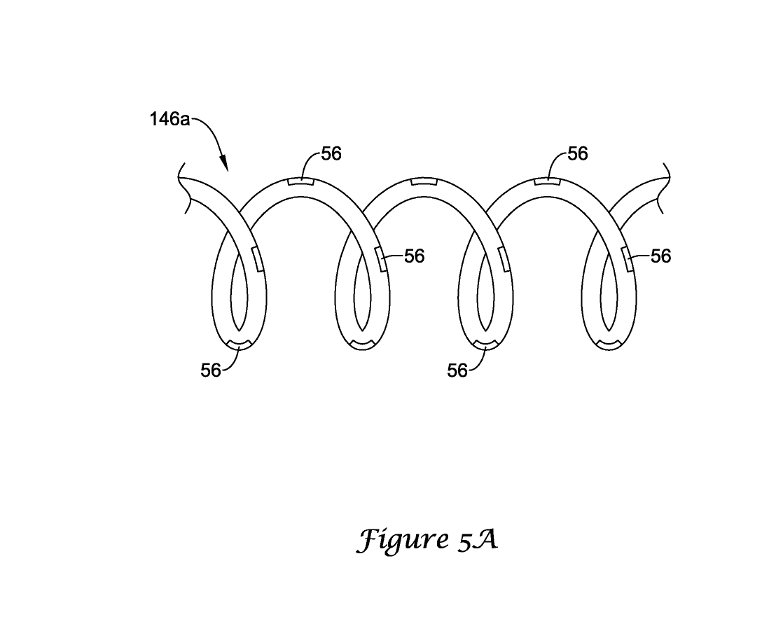

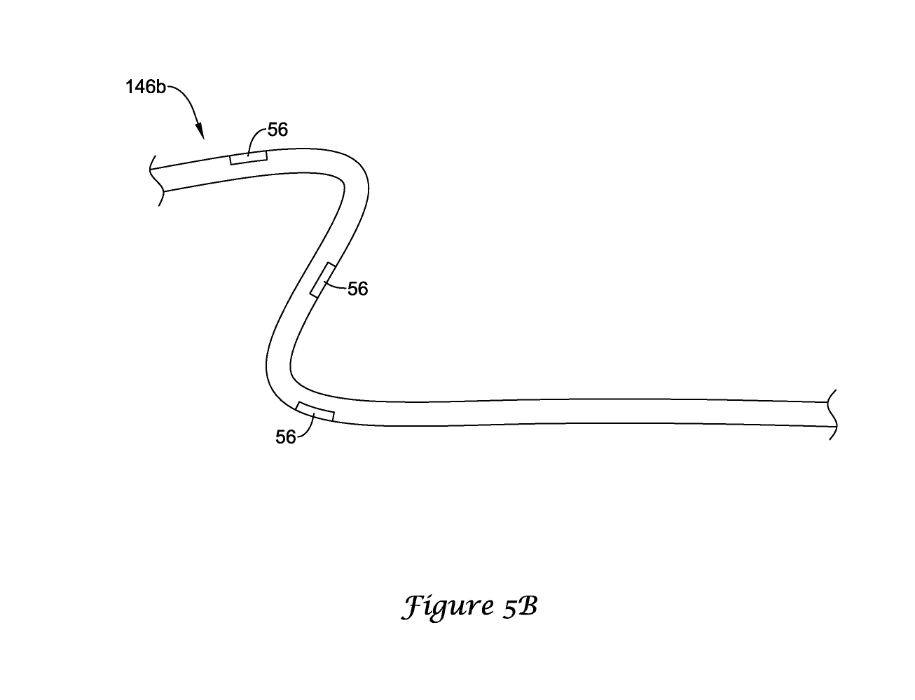

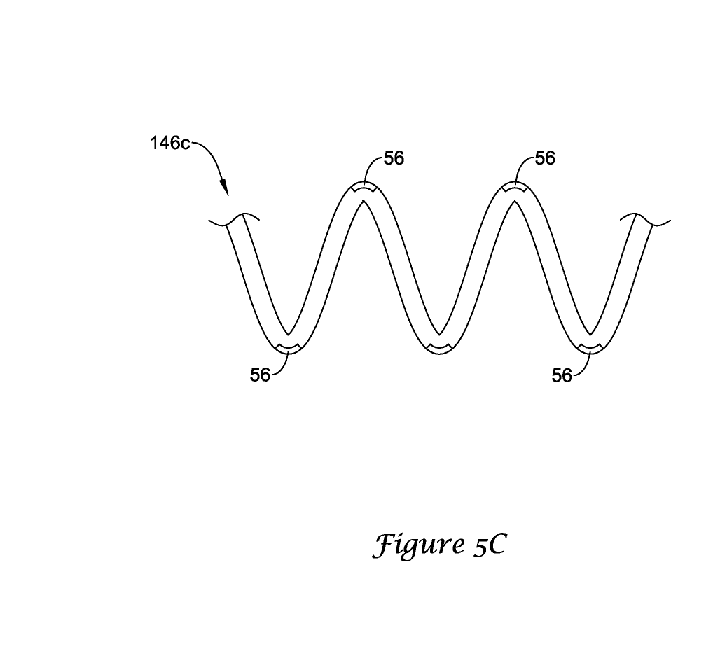

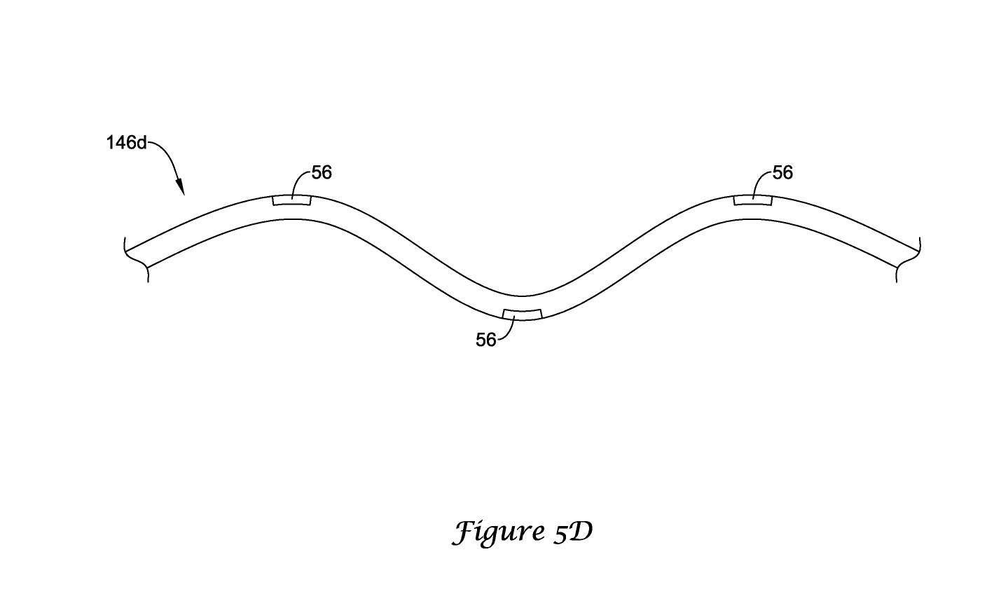

FIGS. 5A-5D are schematic views of several illustrative ablation regions 146a-146d in the second configuration. As shown in the figures, when in the second configuration, each of the ablation regions 146a-146d include at least one curve, bend or turn. The ablation regions 146a-146d are configured such that in the second configuration they have a substantially two-dimensional or three-dimensional shape. According to the illustrative embodiments shown in FIGS. 5A-5D, ablation regions 146a-146d may have a spiral or helical shape (FIG. 5A), a Z or S shape (FIG. 5B), or a generally sinusoidal shape (FIGS. 5C and 5D). As shown in the illustrative embodiments depicted in FIGS. 5A-5D, one or more ablation elements 56 (e.g. electrodes) can be located along a length of each of the ablation regions 146a-146d such that when the ablation regions 146a-146d are in the second configuration, at least two ablation elements 56 are located on opposite sides of the ablation regions 146a-146d so that when the ablation region 146a-146d is deployed in a body lumen or vessel, the ablation elements 56 are positioned near or against opposite walls of the vessel in which the catheter may be deployed. While the ablation elements 56 are shown in the illustrative FIGS. 5A-5D as being located on opposite sides of the ablation regions 146a-146d, it will be generally understood by those of skill in the art that in other embodiments the ablations elements 56 may be placed any number of degrees apart from one another about the circumference of the ablation regions 146a-146d. For example, two or more ablation elements 56 may be spaced apart from one another by approximately 0 degrees to approximately 180 degrees or more particularly, from approximately 0 degrees to approximately 90 degrees about the outer circumference of the ablations regions 146a-146d. The ablations elements 56 can be electrodes, as discussed herein.

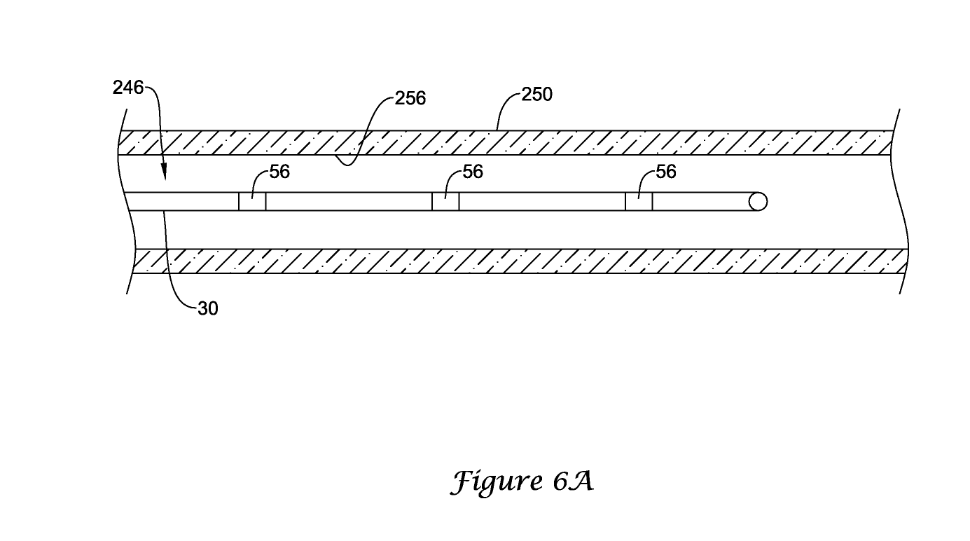

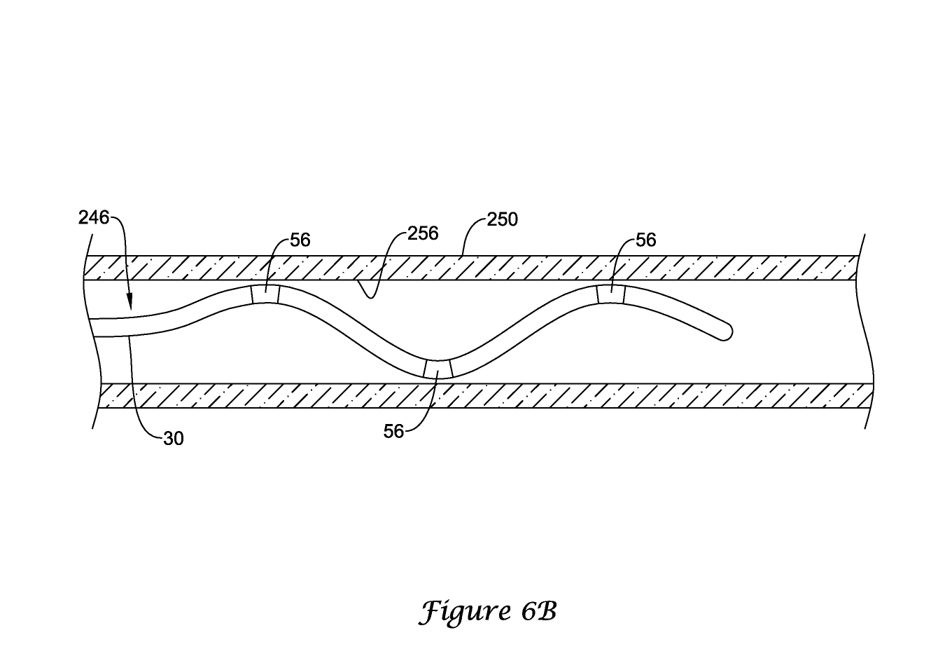

FIGS. 6A and 6B are schematic views of an illustrative ablation region 246 of an exemplary catheter (e.g. catheter 30) during deployment in a body lumen or vessel 250 located adjacent target nerve tissue. In one embodiment, the body lumen or vessel 250 is the renal artery and the target nerve tissue includes a portion or portions of the renal nerves extending along the renal artery. Catheter 30, such as described herein, is delivered to a location within the body lumen or vessel 250 (e.g. renal artery) adjacent the target tissue (e.g. renal nerve tissue). Catheter 30 includes an ablation region 246 according to any one of the embodiments described herein. Once the ablation region 246 is delivered to a site adjacent the target tissue, the ablation region 246 is transitioned from a substantially straight first configuration suitable for delivery (FIG. 6A) to a second configuration including at least one bend, curve or turn such that at least a portion of the ablation region 246 including one or more of electrodes 56 may be positioned in closer proximity to the target tissue (FIG. 6B). The ablation region 246 is transitioned from the first configuration to the second configuration by actuating an actuation member provided with the catheter. In one embodiment, as discussed herein, the actuation member is a pull wire that is attached at or near a distal end of the ablation region 246 that, when actuated in a proximal direction, causes the ablation region 246 to transition from the first configuration to the second configuration. In another embodiment, the actuation member is a sheath that is disposed over the ablation region 246 that, when retracted in a proximal direction to expose the ablation region 246, causes the ablation region 246 to automatically transition from the first configuration to the second configuration.

In the second configuration, as shown in FIG. 6B, the one or more ablation elements 56 (e.g. electrodes) are placed near or in contact with a vessel wall 256 of the body lumen or vessel 250 in which the catheter is deployed such that the one or more ablation elements 56 are placed in closer proximity to the target nerve tissue. While the ablation elements 56 are depicted in the figures as being circumferential bands that may directly contact the vessel wall 256, it will be generally understood that the ablation elements 56 may be recessed from an outer surface of the ablation region 246 and/or may only extend partially around an outer circumference of the ablation region 246. The ablation elements 56 are distributed along a length of the ablation region 246 such that when the ablation region is in the second configuration, as shown in FIG. 6B, the ablation elements 56 are capable of achieving complete circumferential coverage of the body vessel or lumen 250 in which the catheter 30 is deployed, while at the same time being spaced apart from one another longitudinally along its length. As such, when the ablation region 246 is in the second configuration, the ablation elements 56 may be capable of ablating the target nerve tissue at multiple locations along the length and around a circumference of the body lumen or vessel 250 without the need for repositioning the catheter 30 within the body lumen or vessel adjacent the target nerve tissue. Once the ablation region 246 is in the second configuration, sufficient energy to ablate the target nerve tissue can be delivered via the one or more ablation elements 56. In one embodiment, sufficient energy to ablate the target nerve tissue need only be delivered once to achieve the desired result without the need to reposition the catheter 30.

After ablation has occurred, the ablation region 246 is transitioned from the second configuration to the first configuration for repositioning of the catheter 30 within the vessel 250 and/or withdrawal. It will be generally understood that the ablation procedure, as described herein, may be performed under visualization (e.g. fluoroscopy) using techniques known to those of skill in the art.

Although various embodiments of the disclosure are specifically illustrated and described herein, it will be appreciated that modifications and variations of the present disclosure are covered by the above teachings without departing from the spirit and intended scope of the disclosure.

What is claimed is:

1. An intravascular catheter for modulating and/or ablating nerves, the catheter comprising:
an elongated catheter body including an ablation region, the ablation region comprising a flexible portion having a plurality of slots formed therein defining at least one spine extending along a length of the flexible portion and a plurality of ribs extending away from the spine, the ablation region configured to transition from a first configuration suitable for delivery of the catheter to a second configuration having at least one bend, curve or turn suitable for ablating the nerves;
a conductor extending within the elongated catheter body;
two or more ablation elements coupled to the conductor extending within the elongated catheter body and located along the ablation region; and
an actuation member coupled to the ablation region for transitioning the ablation region from the first configuration to the second configuration.

2. The intravascular catheter according to claim 1, wherein the two or more ablation elements are electrodes, wherein each electrode is configured to deliver sufficient RF energy so as to ablate the nerves.

3. The intravascular catheter according to claim 1, wherein the actuation member comprises a pull wire coupled to a distal end of the flexible portion.

4. The intravascular catheter according to claim 1, wherein the actuation member comprises a delivery sheath that is configured to retain the ablation region in the first configuration for delivery and that when withdrawn allows the ablation region to automatically transition from the first configuration to the second configuration.

5. The intravascular catheter according to claim 1, wherein in the first configuration the ablation region is substantially straight and in the second configuration the ablation region forms an elongated spiral.

6. The intravascular catheter according to claim 1, wherein in the first configuration the ablation region is substantially straight and in the second configuration the ablation region comprises at least one S-shaped curve.

7. The intravascular catheter according to claim 1, wherein in the first configuration the ablation region is substantially straight and in the second configuration the ablation region forms a sinusoidal shape.

8. The intravascular catheter according to claim 1, wherein in the first configuration the ablation region is substantially straight and in the second configuration the ablation region forms a Z-shape.

9. The intravascular catheter according to claim 2, wherein at least one of the two or more electrodes is located at a distal end of the catheter body.

10. The intravascular catheter according to claim 2, wherein each of the electrodes extends at least partially around a circumference of the flexible portion.

11. The intravascular catheter according to claim 2, wherein each of the electrodes is recessed from an outer surface of the flexible portion such that they do not contact an artery wall when the ablation region is in the second configuration.

12. The intravascular catheter according to claim 1, wherein the flexible portion comprises a plurality of slots formed therein defining two spines extending along a length of the ablation region, the plurality of ribs extending between the two spines from a first spine to a second spine.

13. The intravascular catheter according to claim 1, further comprising a power and control element electrically coupled to the conductor extending with the catheter body for delivering electrical energy to each of the two or more ablation elements.

14. A method of ablating target nerve tissue from a location within a body vessel, the method comprising:
delivering an intravascular catheter to a location within the body vessel adjacent the target nerve tissue, the catheter comprising an elongated catheter body including an ablation region, the ablation region comprising a flexible portion and configured to transition from a first configuration suitable for delivery of the catheter to a second configuration for ablating target tissue in a circumferential pattern along a length of the body vessel, an electrical conductor extending within the elongated catheter body, and a plurality of ablation elements located along the ablation region and coupled to the conductor extending within the elongated catheter body;
transitioning the ablation region from the first configuration to the second configuration; and
delivering sufficient energy via the ablation elements positioned along the ablation region, wherein the target renal nerve tissue is ablated in a substantially circumferential pattern along the length of the ablation region.

15. The method according to claim 14, wherein transitioning the ablation region from the first configuration to the second configuration comprises actuating a pull wire coupled to the ablation region in a proximal direction.

16. The method according to claim 14, wherein transitioning the ablation region from the first configuration to the second configuration comprises withdrawing a sheath disposed about the ablation region in a proximal direction to expose the ablation region and to allow the ablation region to automatically transition from the first configuration to the second configuration.

17. The method according to claim 14, further comprising transitioning the ablation region from the second configuration to the first configuration for repositioning and/or withdrawal of the catheter within the renal artery.

18. An intravascular catheter for modulating and/or ablating nerves, the catheter comprising:
an elongated catheter body including an ablation region, the ablation region comprising a flexible portion having a plurality of slots formed therein defining at least one spine extending along a length of the flexible portion and a plurality of ribs extending away from the spine, the ablation region configured to transition from a first configuration suitable for delivery of the catheter to a second configuration having at least one bend, curve or turn suitable for ablating the nerves;
a conductor extending within the elongated catheter body;
at least one ablation element coupled to the conductor extending within the elongated catheter body and located along the ablation region; and
an actuation member coupled to the ablation region for transitioning the ablation region from the first configuration to the second configuration.

19. The catheter according to claim 18, wherein the at least one ablation element is located at a distal end of the catheter body.

20. The intravascular catheter according to claim 18, wherein in the first configuration the ablation region is substantially straight and in the second configuration the ablation region forms a sinusoidal shape.
